(19) United States
(12) Patent Application Publication
(10) Pub. No.: US 2015/0164012 A1
(43) Pub. Date: Jun. 18, 2015

(54) EFFECTIVE HEAT TREATMENT FOR THE DEVITALIZATION OF REFERENCE SEED MATERIAL

(71) Applicant: DOW AGROSCIENCES LLC, Indianapolis, IN (US)

(72) Inventors: Shawna K. Embrey, Indianapolis, IN (US); James K. Cruse, Indianapolis, IN (US)

(21) Appl. No.: 14/566,224

(22) Filed: Dec. 10, 2014

Related U.S. Application Data
(60) Provisional application No. 61/916,925, filed on Dec. 17, 2013.

(51) Int. Cl. A01H 3/00

(52) U.S. Cl. CPC A01H 3/00

(57) ABSTRACT

A method for devitalizing a seed includes heating the seed to a temperature from about 95° C. to about 100° C. for a predetermined period, wherein said heating step completely devitalizes the seed.

### CROSS-REFERENCE TO RELATED APPLICATIONS

This application claims the benefit of U.S. Provisional Patent Application Ser. No. 61/916,925, filed Dec. 17, 2013, the disclosure of which is expressly incorporated by reference in its entirety.

### FIELD

The present invention relates to methods for treating a seed sample and in particular to methods for devitalizing a seed sample.

### BACKGROUND AND SUMMARY

Submission of whole seed may be required as part of the regulatory approval process in some jurisdictions, such as for the approval of transgenic seed. It is desirable for an intact whole seed to be devitalized. Devitalization refers to the process of removing the ability of seed to germinate.

One typical devitalization method is to autoclave seed. Typical autoclave methods include treating the seed with high pressure saturated steam having a temperature of 121° C. or greater for about 15-20 minutes. The high temperature used in autoclaving may significantly degrade the DNA of the seed. In addition, autoclaving may result in significant changes to the color of the seed or hull. Discolored seeds may be rejected by regulatory agencies requiring the devitalized reference seed to be visually similar to the germinating seed.

Another typical devitalization method is to hydrate or imbibe a plant seed, followed by freezing the seed by subjecting the hydrated seed to a low temperature. However, this method is typically limited to devitalizing 50 seeds or less at a time. In addition, freezing the imbibed may result in the seed fracturing. Fractured seeds may be rejected by regulatory agencies requiring intact whole seed.

Another devitalization method is to treat seed by subjecting it to a temperature of about 120° C. for 2 hours at ambient pressure. While seeds devitalized in this manner may be suitable for detection of single event specific PCR (polymerase chain reaction) detection, some significant DNA degradation is typically observed.

Some embodiments of the present disclosure include methods for devitalizing a seed. These methods include heating the seed to a temperature greater than 90° C. and less than 120° C. for a predetermined period. In one embodiment, the temperature is from about 95° C. to about 100° C. In some embodiments, the heating step completely devitalizes the seed. In some embodiments, the seed exhibits minimal or no genomic DNA degradation following the heating step.

The above mentioned and other features of the invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIG. 1 illustrates an exemplary method of devitalizing seed.

FIG. 2A shows germinated seed from a non-devitalized soybean sample.

FIG. 2B shows non-germinated seed from a soybean sample devitalized at 95° C.

FIG. 3A shows germinated seed from a non-devitalized corn sample.

FIG. 3B shows non-germinated seed from a corn sample devitalized at 95° C.

FIG. 4A shows germinated seed from a non-devitalized cotton sample.

FIG. 4B shows non-germinated seed from a cotton sample devitalized at 95° C.

FIG. 5A shows germinated seed from a non-devitalized soybean sample.

FIG. 5B shows non-germinated seed from a soybean sample devitalized at 120° C. for 2 hours.

FIG. 6A shows germinated seed from a non-devitalized corn sample.

FIG. 6B shows non-germinated seed from a corn sample devitalized at 120° C. for 2 hours.

FIG. 7A shows germinated seed from a non-devitalized cotton sample.

FIG. 7B shows non-germinated seed from a cotton sample devitalized at 120° C. for 2 hours.

FIG. 8A shows soybeans from a non-devitalized control sample.

FIG. 8B shows soybeans following devitalization at 95° C.

FIG. 9A shows a comparison of transgenic protein levels in corn seeds devitalized at 95° C. and 100° C.

FIG. 9B shows a comparison of transgenic protein levels in soybean seed devitalized at 95° C. and 100° C.

FIG. 9C shows a comparison of transgenic protein levels in soybean seed devitalized at 95° C. and 100° C.

FIG. 10A shows an electrophoresis gel illustrating molecular weight of genomic DNA associated with non-devitalized corn samples and corn samples devitalized at 95° C.

FIG. 10B shows an electrophoresis gel illustrating molecular weight of genomic DNA associated with non-devitalized soybean samples and soybean samples devitalized at 95° C.

FIG. 10C shows an electrophoresis gel illustrating molecular weight of genomic DNA associated with non-devitalized cotton samples and cotton samples devitalized at 95° C.

FIG. 11 shows an electrophoresis gel illustrating the molecular weight of genomic DNA associated with non-devitalized soybean samples and soybean samples devitalized at 120° C. for 2 hours.

### DETAILED DESCRIPTION OF THE DRAWINGS

The embodiments disclosed below are not intended to be exhaustive or to limit the invention to the precise forms disclosed in the following detailed description. Rather, the embodiments are chosen and described so that others skilled in the art may utilize their teachings. While the present disclosure is primarily directed the devitalization of transgenic seed, it should be understood that the features disclosed herein may have application to the treatment of other samples.

Unless stated otherwise, the term “about” as used herein, means plus or minus 10 percent, e.g. about 2.0 includes values between 1.8 and 2.4. When applied to a temperature, the term “about” means plus or minus 2° C., e.g. a temperature of about 100° C. includes values between 98° C. and 102° C.

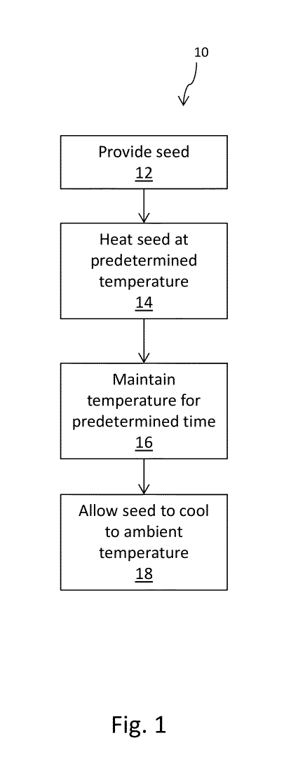

Referring first to FIG. 1, a method 10 of devitalizing seed is provided. Seed is provided or received, as shown in block 12. Exemplary seed include corn or maize (Zea Mays), cotton (Gossypium), and soybean (Glycine max). In one embodiment, the seed provided in block 12 is a single seed. In one embodiment, the seed provided in block 12 is a plurality of seeds. In one embodiment, the plurality of seeds provided in block 12 is at least 0.5 kilograms, 1 kilogram, 5 kilograms, 10 kilograms, 30 kilograms, or more, or within any range defined between any two of the foregoing values.

The seed is heated at a predetermined temperature, as shown in block 14. In one embodiment, the temperature is greater than 90° C., but less than 120° C. In one embodiment, the temperature is as low as 91° C., 95° C., 97° C., as great as 100° C., 105° C., 110° C., 115° C., 119° C., or within any range defined between any two of the foregoing values. In one embodiment, the temperature is from about 95° C. to about 100° C. In one embodiment, the temperature is 95° C. In another embodiment, the temperature is 100° C.

The seed is exposed to the temperature for a predetermined time, as shown in block 16. In one embodiment, the predetermined time is as short as 4 hours, 8 hours, 12 hours, 24 hours, 3 days as long as 6 days, 7 days, 8 days, 9 days, 12 days, 14 days, or longer, or within any range defined between any two of the foregoing values. In one embodiment, the predetermined time is at least about 24 hours. In one embodiment, the predetermined time is about 24 hours to about 9 days.

The seed is then allowed to cool from the elevated temperature, as shown in block 18. In one embodiment, the seed is allowed to cool to ambient temperature. In one embodiment, the seed is allowed to cool without active cooling. In one embodiment, the seed is actively cooled, such as by exposure of the seed to a gas or liquid.

Germination was tested using sections 6-1 through 6-60 of 2010 AOSA Rules for Testing Seeds, Association of Official Seed Analysts, Inc. (AOSA), Moline, Ill.

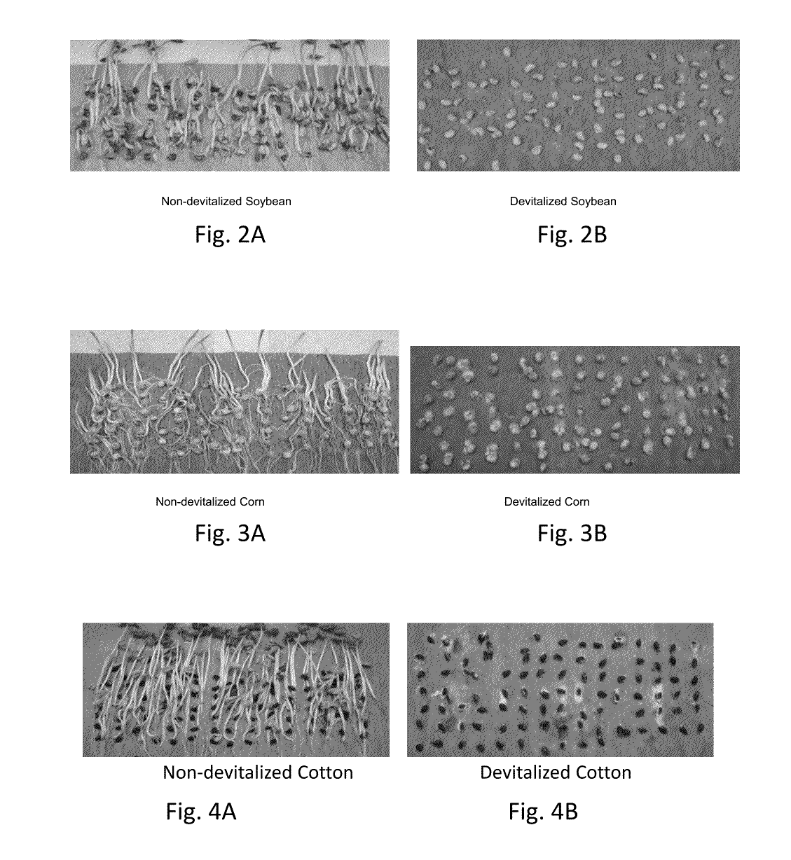

Referring next to FIGS. 2-4, the method illustrated in FIG. 1 devitalized the treated seed. FIG. 2A shows germinated seed from an untreated soybean sample. FIG. 2B shows that a similar soybean sample treated at 95° C. did not germinate. FIG. 3A shows germinated seed from an untreated corn sample. FIG. 3B shows that a similar corn sample treated at 95° C. did not germinate. FIG. 4A shows germinated seed from an untreated cotton sample. FIG. 3B shows that a similar cotton sample treated at 95° C. did not germinate.

In one embodiment, devitalizing the seed produces seeds having a germination rate of less than 1%. In one embodiment, the germination rate following the devitalization treatment is about 0%. In a more particular embodiment, the germination rate is 0%.

In one embodiment, the seed is selected include corn or maize (Zea Mays), cotton (Gossypium), and soybean (Glycine max). Other suitable seed, including rapeseed, rice, and canola, may also be used.

In one embodiment, the seed is transgenic. In one embodiment, the transgene encodes one or more herbicide resistant trait, such as the aryloxyalkanoate dioxygenase-12 protein (AAD-12). In one embodiment the transgene encodes one or more Bacillus thuringiensis (Bt) related traits, such as the Cry1F protein (Cry1F), Cry1Ac protein (Cry1Ac), and phosphinothricin-N-acetyl transferase protein (PAT). In one embodiment, the transgene encodes one or more of the Cry34Ab1 protein, the Cry35Ab1 protein, and the arylloxyalkanoate dioxygenase-1 (AAD-1) protein. Other suitable transgenic seed may also be used.

In one embodiment, devitalizing the seed produces little or no degradation of a typical protein. In some embodiments, the typical protein has a molecular weight as low as about 14,000 Dalton, 20,000 Dalton, as high as about 130,000 Dalton, 250,000 Dalton, or within any range defined between any two of these values. In some embodiments, the typical protein is encoded by at least one transgene. In some embodiments, the typical protein is selected from the group consisting of aryloxyalkanoate dioxygenase-12 (AAD-12), Cry1F (Cry1F), Cry34Ab1, Cry35Ab1, Cry1Ac, phosphinothricin-N-acetyl transferase (PAT), and arylloxyalkanoate dioxygenase-1 (AAD-1) proteins.

In one embodiment, the degradation of the typical protein is determined by comparing the concentration of that protein, such as determined by an ELISA quantification, from a treated sample with the concentration of the same protein in a similar untreated control seed. In some embodiments, the degradation as measured by the difference in protein concentration, is as little as 0%, 10%, 25%, as great as 50%, 75%, 80%, or within any range defined between any two of the foregoing values. In one embodiment, the protein is readily detectable using an ELISA quantification method following devitalization.

In one embodiment, devitalizing the seed produces little or no genomic DNA degradation. In one embodiment, the amount of genomic DNA degradation is determined by comparing the molecular weight distribution of a sample extracted from a treated seed with a sample extracted from a similar untreated control. An exemplary method of comparing the molecular weight distribution is with an agarose gel, where a wider band indicates a wider distribution of molecular weights associated with a higher level of genomic DNA degradation. In one embodiment there is no genomic DNA degradation.

In one embodiment, the seed is maize, and the seed is heated to a temperature greater than 90° C., but less than 120° C. for a period of about 4 hours or longer. In a more particular embodiment, the temperature is as low as 91° C., 95° C., 100° C., as great as 105° C., 110° C., 115° C., 119° C., or within any range defined between any two of the foregoing values, and the predetermined time is as 4 hours, 8 hours, 12 hours, 24 hours, 3 days as long as 6 days, 7 days, 8 days, 9 days, 12 days, 14 days, or longer or within any range defined between any two of the foregoing values. In another more particular embodiment, the temperature is about 95° C. to about 100° C. and the predetermined time is about 24 hours. In still another more particular embodiment, the temperature is about 95° C. and the predetermined time is about 24 hours. In yet still another more particular embodiment, the temperature is about 100° C. and the predetermined time is 24 hours.

In one embodiment, the seed is cotton, and the seed is heated to a temperature greater than 90° C., but less than 120° C. for a period of about 4 hours or longer. In a more particular embodiment, the temperature is as low as 91° C., 95° C., 100° C., as great as 105° C., 110° C., 115° C., 119° C., or within any range defined between any two of the foregoing values, and the predetermined time is as short as 4 hours, 8 hours, 12 hours, 24 hours, 3 days as long as 6 days, 7 days, 8 days, 9 days, 12 days, 14 days, or longer, or within any range defined between any two of the foregoing values. In another more particular embodiment, the temperature is about 95° C. to about 100° C. and the predetermined time is about 9 days. In still another more particular embodiment, the temperature is about 95° C. and the predetermined time is about 9 days. In yet still another more particular embodiment, the temperature is about 100° C. and the predetermined time is about 9 days.

In one embodiment, the seed is soybean, and the seed is heated to a temperature greater than 90° C., but less than 120° C. for a period of about 4 hours or longer. In a more particular embodiment, the temperature is as low as 91° C., 95° C., 100° C., as great as 105° C., 110° C., 115° C., 119° C., or within any range defined between any two of the foregoing values, and the predetermined time is as 4 hours, 8 hours, 12 hours, 24 hours, 3 days as long as 6 days, 7 days, 8 days, 9 days, 12 days, 14 days, or longer, or within any range defined between any two of the foregoing values. In another more particular embodiment, the temperature is about 95° C. to about 100° C. and the predetermined time is about 6 days. In still another more particular embodiment, the temperature is about 95° C. and the predetermined time is about 6 days. In yet still another more particular embodiment, the temperature is about 100° C. and the predetermined time is about 6 days.

### EXAMPLES

### Germination Testing and Seed Integrity

Devitalization was attempted on different seed using temperatures between 60° C. and 120° C. Devitalization was determined by attempting to germinate 400-3000 seeds according to section 6-1 through 6-60 of the 2010 AOSA Rules for Testing Seeds, Association of Official Seed Analysts, Inc. (AOSA), Moline, Ill.

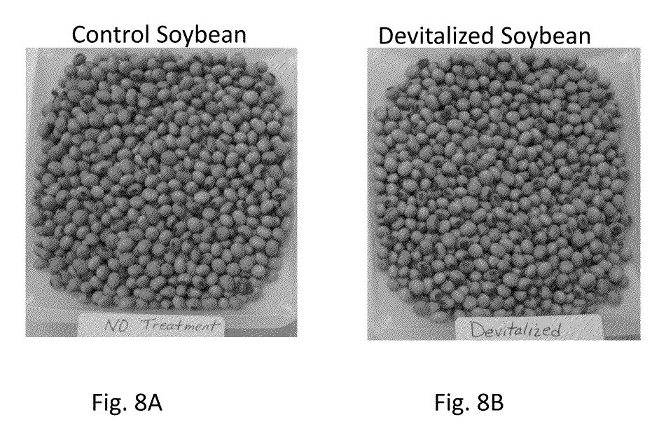

Each sample was visually inspected for seed integrity. No fractured samples were observed. No discolored samples were observed at temperatures below 120° C., such as the 95° C. treated soybean shown in FIG. 8B compared to the untreated control shown in FIG. 8A. Discoloration was observed in corn at 120° C., suggesting that the discoloration is due to the seed essentially toasting at this temperature.

### Protein Presence and Quantification

Protein presence and quantification were performed by comparing a sample from the treated seed with a sample from an unheated control. For each sample, a transgenic seed was ground and analyzed for the presence of a transgene using a commercially available enzyme-linked immunosorbent assay (ELISA) kit. For maize, the ground seed was analyzed for the presence of the Cry1F insecticidal crystal protein. For cotton, the ground seed was analyzed for the presence of Cry1F insecticidal crystal protein. For soybean, the ground seed was analyzed for the presence of AAD-12 (aryloxyalkanoate dioxygenase 12).

Each sample (treated and untreated seeds and conventional control) was ground using two steel ball bearings in a Geno-Grinder for 3 minutes at 1500 strokes/minute. Approximately 15 mg and 120 mg samples of each tissue were extracted from the ground samples with a buffer solution. The extract was centrifuged; the aqueous supernatant was collected, diluted and assayed using a protein ELISA kit specific to the particular protein being investigated.

For each sample, an aliquot of the diluted sample was incubated with enzyme-conjugated anti-X (Cry1F or AAD-12) protein antibody in the wells of an anti-X (Cry1F or AAD-12) antibody coated plate to form an antibody-protein-antibody/enzyme conjugate sandwich. Both antibodies in the sandwich pair capture the protein of interest in the sample. At the end of the incubation period, the unbound reagents are removed from the plate by washing with PBST (phosphate buffered saline)

The presence of the Cry1F or AAD-12 protein was detected by incubating the antibody-bound enzyme conjugate with an enzyme substrate, generating a colored product. Since the target protein was bound in the antibody sandwich, the level of color development was proportional to the concentration of the protein in the sample (i.e., lower protein concentrations result in lower color development). The absorbance at either 450 nm or 450 minus 650 was measured using a spectrophotometric plate reader and compared to a standard curve to obtain quantitation of the transgenic proteins in the seed tissue extracts.

### Analysis of Genomic DNA Integrity

Genomic DNA was isolated from soy, maize, and cotton seeds using a genomic DNA extraction kit provided by Genetic ID NA, Inc. (FASTID Cat: K1-0001-0200). Approximately 200 mg of tissue of each sample (treated and untreated seeds and conventional control) were suspended in a buffer solution which lysed the cells of the samples and solubilized the proteins, DNA, and other cellular constituents. Proteinase K was added to digest protein in the samples.

A chloroform purification step was then performed to separate the digested proteins from the supernatant containing the DNA. A genomic DNA binding buffer was then added to the DNA-containing supernatant and the mixture was passed through a column that binds the DNA.

Contaminants were washed from the column with a specially formulated wash buffer and a series of ethanol washes. The DNA was eluted from the column using a 1×Tris EDTA (TE) buffer. The DNA was quantified using PicoGreen (Invitrogen, Carlsbad, Calif.) and 200 ng of genomic DNA from each of the treated and non-treated seed samples were independently loaded into an agarose gel and analyzed to determine the level of degradation to the genomic DNA that was extracted.

### Example 1

### Temperature of 60° C. to 90° C.

Maize, cotton, and soybean were heated to the temperature as shown in Table 1 for the period shown. DNA integrity was preserved at temperatures of 60° C., 80° C., and 90° C. At temperatures of 60° C. and 80° C., Germination of the samples was observed even after a month of heat treatment.

TABLE 1
Maize, cotton, and soybean at temperatures 60° C. to 90° C.

| Seed type | Temperature | Time | Germination |
|---|---|---|---|
| Maize | 60° C. | 1 month | Not acceptable* |
| Cotton | 60° C. | 1 month | Not acceptable* |
| Soybean | 60° C. | 1 month | Not acceptable* |
| Maize | 80° C. | 1 month | Not acceptable* |
| Cotton | 80° C. | 1 month | Not acceptable* |
| Soybean | 80° C. | 1 month | Not acceptable* |
| Maize | 90° C. | 1 month | >1% |
| Cotton | 90° C. | 1 month | >1% |
| Soybean | 90° C. | 1 month | >1% |

*Germination results of treated seeds showed little difference from the non- devitalized seeds and were not considered acceptable after 1 month of testing.

### Example 2

### Temperature of 95° C. to 100° C.

Following the encouraging results at 90° C., the temperature was increased to further remove moisture from the seed. Maize, cotton, and soybean were heated to the temperature as shown in Table 2 for the period shown. The seeds showed no or minimal degradation at the tested temperature and times. At temperatures of 95° C. and 100° C., complete devitalization was achieved and no germination was seen.

As shown in FIGS. 8A and 8B, the soybeans treated at 95° C. (FIG. 8B) did not discolor compared to a similar untreated control sample (FIG. 8A).

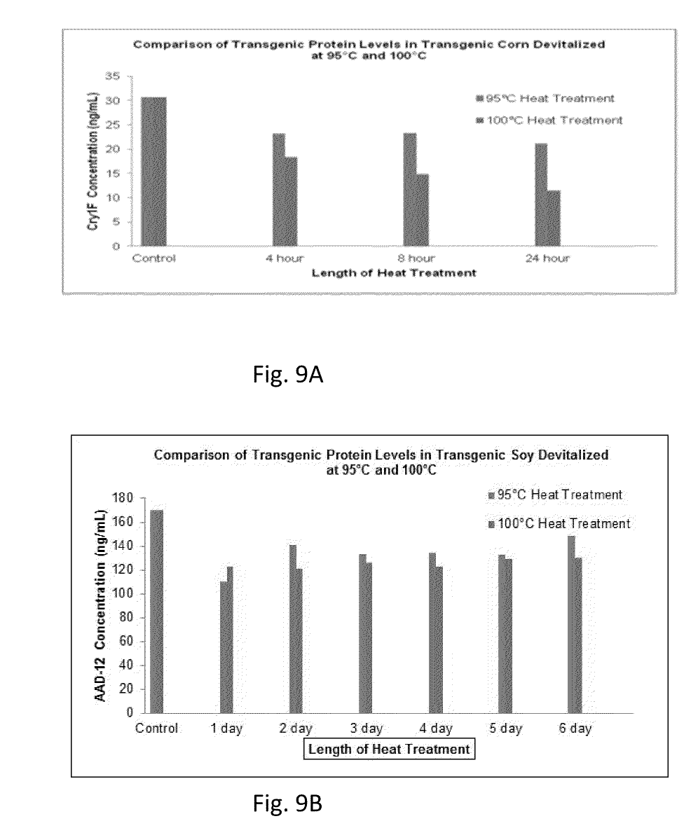

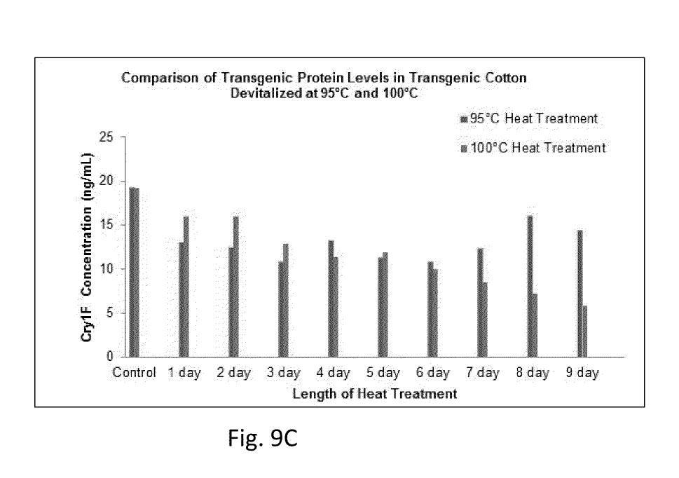

ELISA analysis of the Cry1F and AAD-12 proteins in maize, cotton and soybean determined that each protein was readily detectable as compared to the unheated conventional control. Referring to FIGS. 9A-9C, the Cry1F protein was readily detected in quantifiable amounts following treatment at 95° C. and 100° C. at times from 4 to 24 hours for maize (FIG. 9A) and 1 day to 9 days for cotton (FIG. 9C). The AAD-12 protein was readily detected in quantifiable amounts following treatment at 95° C. and 100° C. at times from 1 to 6 days for soybeans (FIG. 9B).

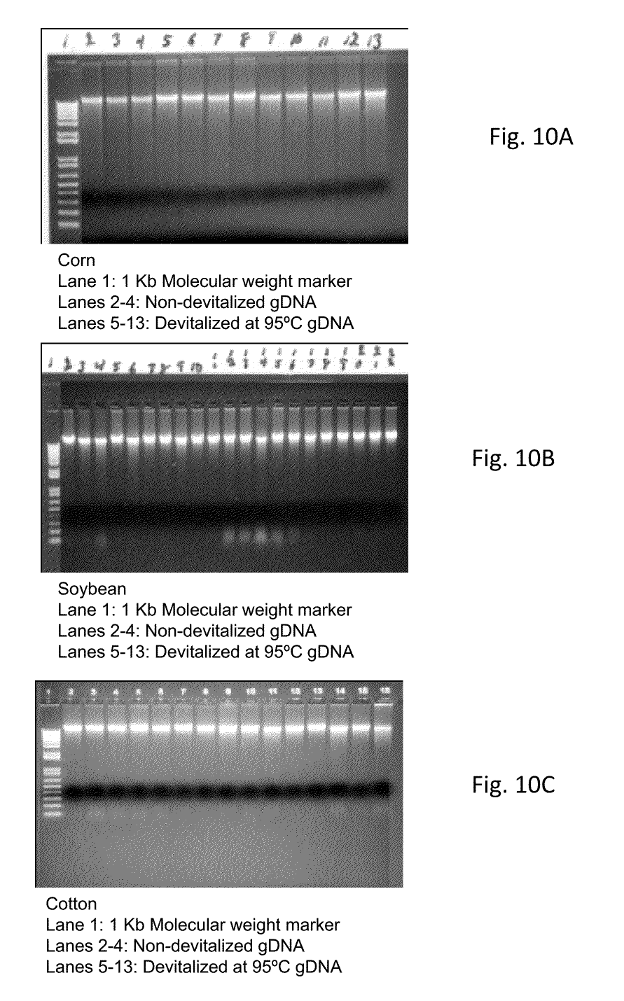

Genomic DNA analysis determined that that the devitalization process resulted in little to no genomic DNA degradation. Referring to FIG. 10A, a gel showing the molecular weight of genomic DNA from non-devitalized maize samples in lanes 2-4 and devitalized maize samples in lanes 5-13. Referring to FIG. 10B, a gel showing the molecular weight of genomic DNA from non-devitalized soybean samples in lanes 2-4 and devitalized soybean samples in lanes 5-13. Referring to FIG. 10C, a gel showing the molecular weight of genomic DNA from non-devitalized cotton samples in lanes 2-4 and devitalized cotton samples in lanes 5-13. A wider band indicates a wider range of molecular weights associated with a higher level of genomic DNA degradation. The bands for the samples treated at 95° C. (lanes 5-13) were observed to be very similar to those of the untreated control samples (lanes 2-4) for each of the seed types tested. It was determined that there was minimal, if any, difference in the degradation of the genomic as compared to the conventional control.

TABLE 2
Maize, cotton, and soybean at temperatures 95° C. to 100° C.

| | | | | Number of seeds |
|---|---|---|---|---|
| Seed type | Temperature | Time | | germinated |
| Maize | 95° C. | 24 | hours | 0 of 3000 |
| Cotton | 95° C. | 9 | days | 0 of 3000 |
| Soybean | 95° C. | 6 | days | 0 of 3000 |
| Maize | 100° C. | 24 | hours | 0 of 3000 |
| Cotton | 100° C. | 9 | days | 0 of 3000 |
| Soybean | 100° C. | 6 | days | 0 of 3000 |

### Example 3

### Temperature of 120° C.

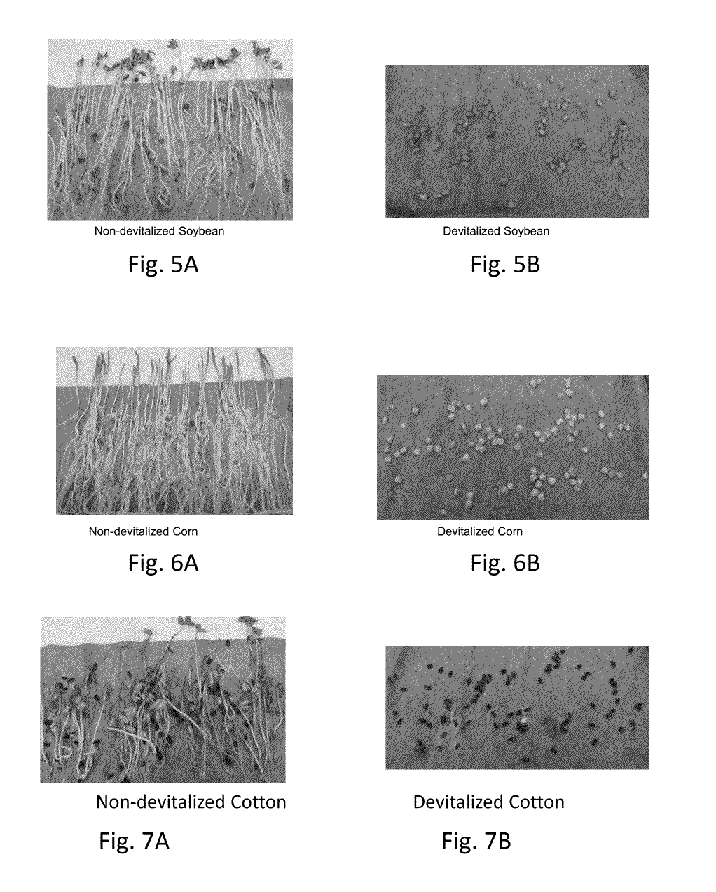

The temperature was increased further to 120° C., and maize, cotton, and soybean were heated to the temperature as shown in Table 3 for the period shown. FIG. 5A shows germinated seed from an untreated soybean sample. FIG. 5B shows that a similar soybean sample treated at 120° C. for 2 hours did not germinate. FIG. 6A shows germinated seed from an untreated corn sample. FIG. 6B shows that a similar corn sample treated at 120° C. for 2 hours did not germinate. FIG. 7A shows germinated seed from an untreated cotton sample. FIG. 7B shows that a similar cotton sample treated at 120° C. for 2 hours did not germinate.

TABLE 3
Maize, cotton, and soybean at temperature 120° C.

| | | | | DNA |
|---|---|---|---|---|
| Seed type | Temperature | Time | Germination | degradation |
| Soybean | 120° C. | 2 hours | None | Severe |

A temperature of 120° C. provided complete devitalization and no germination was observed. However, DNA degradation of the seeds was observed

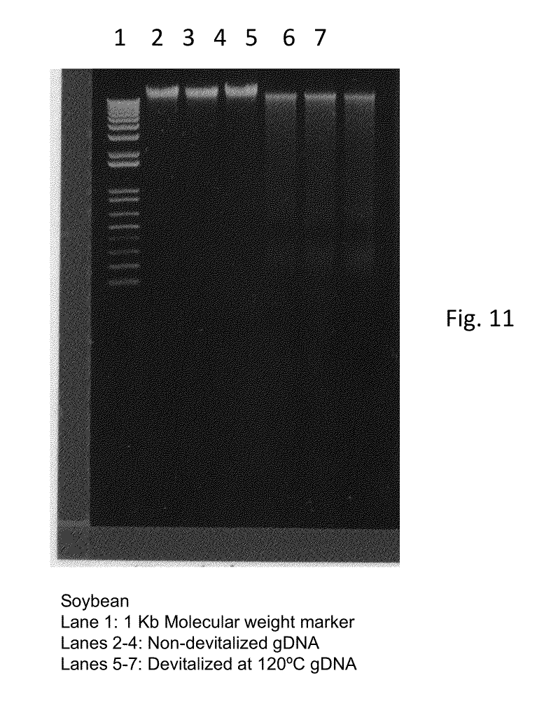

As shown in Table 3, the genomic DNA of soybean treated for 2 hours at 120° C. was severely degraded and was considered unacceptable for use as reference material in DNA assays. Referring to FIG. 11, a gel showing the molecular weight of genomic DNA from non-devitalized soybean samples in lanes 2-4 and soybean samples in lanes 5-13. The bands for the samples treated for 2 hours at 120° C. (lanes 5-7) were observed to be significantly wider compared to those of the untreated control samples (lanes 2-4). This indicated significant DNA degradation of the treated samples.

While this invention has been described as relative to exemplary designs, the present invention may be further modified within the spirit and scope of this disclosure. Further, this application is intended to cover such departures from the present disclosure as come within known or customary practice in the art to which this invention pertains.

1. A method for devitalizing a seed comprising:
heating the seed to a temperature from about 95° C. to about 100° C. for a period of about 4 hours to about 14 days, wherein said heating step completely devitalizes the seed.

2. The method of claim 1, wherein the temperature is about 95° C.

3. The method of claim 1, wherein the temperature is about 100° C.

4. The method of claim 1, wherein the seed following the heating step has a concentration of a typical protein readily detectable by an ELISA quantification method.

5. The method of claim 4, wherein the typical protein has a molecular weight of about 20,000 Dalton to about 130,000 Dalton.

6. The method of claim 4, wherein the typical protein has a molecular weight of about 14,000 Dalton to about 250,000 Dalton.

7. The method of claim 4, wherein the typical protein is a protein is selected from the group consisting of: AAD-12, Cry1F, Cry1Ac, PAT, Cry34Ab1, Cry35Ab1, and AAD-1.

8. The method of claim 1, wherein the seed exhibits minimal or no genomic DNA degradation following said heating step.

9. The method of claim 1, wherein the seed has no genomic DNA degradation following said heating step compared to a similar unheated seed.

10. The method of claim 1, wherein the seed is transgenic.

11. The method of claim 1, wherein the seed is maize.

12. The method of claim 11, wherein the predetermined period is from about 4 hours to about 24 hours.

13. The method of claim 1, wherein the seed is cotton.

14. The method of claim 13, wherein the predetermined period is about 1 day to about 9 days.

15. The method of claim 1, wherein the seed is soybean.

16. The method of claim 15, wherein the predetermined period is about 1 day to about 6 days.

17. A method for devitalizing a sample of seeds comprising:
heating the seed to a temperature from about 95° C. to about 100° C. for a predetermined period of about 4 hours to about 14 days;
wherein the sample of seeds has a mass of at least 0.5 kilograms, and said sample of seeds has a germination rate of less than 1%.

18. The method of claim 17, wherein the seeds are maize seeds and the predetermined period is about 4 hours to about 24 hours.

19. The method of claim 17, wherein the seeds are cotton seeds and the predetermined period is about 1 day to about 9 days.

20. The method of claim 17, wherein the seeds are soybean seeds and the predetermined period is about 1 day to about 6 days.
